(19) United States
(12) Patent Application Publication
(10) Pub. No.: US 2004/0121991 A1
(43) Pub. Date: Jun. 24, 2004

(54) Dehydroepiandrosterone (DHEA) congeners for prevention and/or treatment of ulcers

(76) Inventors: Barbara A. Araneo, Salt Lake City, UT (US); Ned M. Weinshenker, Salt Lake City, UT (US); John R. Gebhard, Salt Lake City, UT (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/442,988

(22) Filed: May 22, 2003

Related U.S. Application Data
(60) Provisional application No. 60/434,733, filed on Dec. 20, 2002.

(51) Int. Cl. A61K 31/56

(52) U.S. Cl. 514/169

(57) ABSTRACT

The present invention is related to acute therapeutic uses of dehydroepiandrosterone (DHEA) congeners. These uses include methods for treating or preventing ulcers which comprise administering to a subject either at risk or in need thereof having an ulcer a therapeutic amount of DHEA congener.

### CROSS-REFERENCE TO RELATED APPLICATION

The present application is related to and claims priority under 35 U.S.C. §119(e) to U.S. provisional patent application Serial No. 60/434,733 filed 20 Dec. 2002, incorporated herein by reference.

### BACKGROUND OF THE INVENTION

The present invention relates to therapeutic uses of dehydroepiandrosterone (DHEA) congeners as effective anti-ulcer agents. These uses include the treatment and/or prevention of both skin and mucosal ulcers. Mucosal ulcers include lesions of the oral, gastric, peptic, intestinal, colonic and rectal tissue; whereas skin ulcers include both lesions of the cutaneous tissue and deep venous tissue. In accordance with the present invention, these conditions are prevented or treated by administering dehydroepiandrosterone (DHEA) or chemical derivatives and congeners of DHEA.

The publications and other materials used herein to illuminate the background of the invention, and in particular cases, to provide additional details respecting the practice, are incorporated by reference, and for convenience are numerically referenced in the following text and respectively grouped in the appended bibliography.

The medical definition of ulcer is a transmural lesion of the skin or mucous membranes marked by inflammation, necrosis, and sloughing of the damaged tissue. Ulcers are distinct from scald water, chemical or electrical bums and from wounds caused by abrasion, puncture and incision. Ulcers result from inflammatory action on a tissue culminating in loss of the upper epithelium and certain amount of the subepithelial layers. Refer to FIGS. 1-4 which show illustrations of normal skin, ulcerated skin, normal intestinal mucosa and ulcerated intestine, respectively.

Oral Mucositis. Oral ulcerative mucositis is a common, painful, dose-limiting toxicity of drug and radiation therapy for cancer. The disorder is characterized by breakdown of the epithelium, the submucosal tissue and the vascular network, in the oral cavity to form ulcerative lesions. Mucositis occurs to some degree in more than one third of patients receiving anti-neoplastic drug therapy. The frequency and severity are significantly greater among patients who are treated with induction therapy for leukemia or with many of the conditioning regimens for bone marrow transplant. Among these individuals, moderate to severe mucositis occurs in more than three-quarters of patients. Moderate to severe mucositis occurs in virtually all patients who receive radiation therapy for tumors of the head and neck.

Clinically, mucositis progresses through several stages: (1) Inflammation accompanied by painful mucosal erythema, which can respond to local anesthetics; (2) Painful ulceration with pseudomembrane formation and, in the case of myelosuppressive treatment, potentially life-threatening sepsis, requiring antimicrobial therapy (pain is often of such intensity as to require parenteral narcotic analgesia); and (3) spontaneous healing, occurring about 2-3 weeks after cessation of anti-neoplastic therapy. Standard therapy for mucositis is predominantly palliative, including application of topical analgesics such as lidocaine and/or systemic administration of narcotics and antibiotics. Mucositis includes e.g., stomatitis/oral mucositis, gastrointestinal mucositis, nasal mucositis, and proctitis. Currently, there is no approved treatment for mucositis.

Stomatitis. Inflammation and ulceration of the mouth caused by infection, systemic disease, or a physical agent, other than cancer therapy-related oral mucositis, are called stomatitis. In bacterial infections, usually, the causative agent is streptococci. However other microbes have been implicated in the etiology of the disease, including but not limited to Mycobacterium tuberculosis, Syphilis, Neisseria gonorrhea, Cervicofacial actinomycosis, Pellagra, and both herpes simplex virus Type I and II.

Other causes of Stomatitis. Hemorrhagic oral lesions may occur in erythema multiforme (see below), scurvy, leukemia, thrombocytopenic purpura, and platelet disorders. B vitamins or vitamin C deficiencies, iron-deficiency anemia and dry mouth predisposes the mouth to stomatitis. Oral erythema multiforme is acutely painful stomatitis characterized by diffuse hemorrhagic lesions of the lips and oral mucosa accompanied by fever, malaise, arthralgia. That the lesions are hemorrhagic and the symptoms are systemic shows ulceration within the subepithelial tissue and systemic inflammatory activity, similar to oral mucositis.

Treatment of these various forms of stomatitis includes systemic analgesics or anesthetics.

Gastritis. Inflammation of the gastric mucosa, gastritis, can also be classified according to the site of involvement within the stomach (i.e., cardia, corpus, antrum), and further classified as acute or chronic.

Peptic Ulcers. Peptic ulcers are an excoriated segment of the GI mucosa, typically in the stomach (gastric ulcer) or first few centimeters of the duodenum (duodenal ulcer), which involves the muscularis mutcosae. Ulcers may range in size from several millimeters to several centimeters. Ulcers are delineated from erosions by the depth of penetration; erosions are superficial causing denuding of the epithelium, leaving the subepithelial layers intact. A peptic ulcer may penetrate the wall of the stomach or duodenum and enter the adjacent confined space (lesser sac) or organ (e.g., pancreas, liver). Treatment of gastric and duodenal ulcers had previously focused on neutralizing or decreasing gastric acidity. However, attention has shifted toward eradication of Heliobacter pylori. Antibiotic treatment is used in all H. pylori-infected patients with acute ulcers. H2 blockers have a role as antisecretory drugs in an anti-H. pylori regimen. Certain prostaglandins (especially misoprostol) can inhibit acid secretion and enhance mucosal defense. Sucralfate is a sucrose-aluminum complex that promotes ulcer healing possibly by inhibition of pepsin-substrate interaction, stimulation of mucosal prostaglandin production, and binding of bile salts. Antacids give symptomatic relief, promote ulcer healing, and reduce recurrence.

Crohn's Disease. Crohn's disease (regional enteritis, granulomatous ileitis or ileocolitis) is a nonspecific chronic transmural inflammatory disease that most commonly affects the distal ileum and colon but may occur in any part of the GI tract. The fundamental cause of Crohn's disease is unknown. Evidence suggests that a genetic predisposition leads to an unregulated intestinal inflammatory response to an environmental, dietary, or infectious agent. Cigarette smoking seems to contribute to the development or exacerbation of Crohn's disease. The earliest mucosal lesion of Crohn's disease is crypt injury in the form of inflammation (cryptitis) and crypt abscesses, which progress to tiny focal aphthoid ulcers. Transmural spread of inflammation leads to lymphedema and bowel wall thickening, which may eventually result in extensive fibrosis. No cure is known. Cramps and diarrhea may be relieved by oral administration of anticholinergics. Sulfasalazine and mesalamine (5-aminosalicylic acid), the active moiety, primarily benefits patients with mild to moderate colitis and ileocolitis but has some efficacy in ileitis as well and may also maintain remission. Corticosteroid therapy treats the acute stages of Crohn's disease by dramatically reducing fever and diarrhea, relieving abdominal pain and tenderness, and improving the appetite and sense of well-being. Although as little as 5 or 10 mg/day of prednisone may help control symptoms in some patients, long-term corticosteroid therapy often does more harm than good. Corticosteroids should be avoided when obvious infections (e.g., fistulas, abscesses) are present. The antimetabolites, azathioprine and 6-mercaptopurine, are effective as long-term therapy for Crohn's disease. High-dose cyclosporine has demonstrated benefits in inflammatory and fistulous disease, but its long-term use is contraindicated by multiple toxicities. Infliximab, a monoclonal antibody that inhibits tumor necrosis factor, can be given IV for moderate to severe Crohn's disease (especially fistulous disease) refractory to other treatments; long-term efficacy and side effects remain to be determined.

Ulcerative Colitis. Ulcerative colitis is a chronic, inflammatory, and ulcerative disease arising in the colonic mucosa, characterized most often by bloody diarrhea.

Pathologic changes begin with degeneration of the reticulin fibers beneath the mucosal epithelium, occlusion of the subepithelial capillaries, and progressive infiltration of the lamina propria with plasma cells, eosinophils, lymphocytes, mast cells, and PMNs. Crypt abscesses, epithelial necrosis, and mucosal ulceration ultimately develop. The disease usually begins in the rectosigmoid and may extend proximally, eventually involving the entire colon, or it may involve most of the large bowel at once. Ulcerative proctitis, which is localized to the rectum, is a very common and more benign form of ulcerative colitis. It is often refractory to treatment and undergoes late proximal spread in about 20 to 30% of cases.

A flare-up of colitis is noted by the appearance of bloody diarrhea of varied intensity and duration, interspersed with asymptomatic intervals. Usually an attack begins insidiously, with increased urgency to defecate, mild lower abdominal cramps, and blood and mucus in the stools. However, an attack may be acute and fulminant, with sudden violent diarrhea, high fever, signs of peritonitis, and profound toxemia. Some cases develop following a documented infection (e.g., amebiasis, bacillary dysentery).

Bleeding is the most common local complication. Another particularly severe complication, toxic colitis, occurs when transmural extension of ulceration results in localized ileus and peritonitis. As toxic colitis progresses, the colon loses muscular tone and begins to dilate within hours or days. Plain x-rays of the abdomen show intraluminal gas accumulated over a long, continuous, paralyzed segment of colon—a result of lost muscle tone.

Toxic megacolon (or toxic dilation) exists when the diameter of the transverse colon exceeds 6 cm. The severely ill patient has a fever to 40° C. (104° F.), leukocytosis, abdominal pain, and rebound tenderness. This condition usually occurs spontaneously in the course of especially severe colitis, but some cases may be precipitated by overzealous use of narcotic or anticholinergic antidiarrheal drugs. Treatment must be given in the early stages, preferably before full-blown megacolon occurs, to avert dangerous complications (eg, perforation, generalized peritonitis, septicemia). With prompt, effective treatment, the mortality rate can be held at <4% but may be >40% if perforation occurs.

Major perirectal complications, such as those in granulomatous colitis (e.g., fistulas, abscesses), do not occur.

There are a variety of treatments for colitis. Avoiding raw fruits and vegetables limits mechanical trauma to the inflamed colonic mucosa and may lessen symptoms. A milk-free diet may help but need not be continued if no benefit is noted. An anticholinergic drug or loperamide 2.0 mg or diphenoxylate 2.5 mg po bid to qid is indicated for relatively mild diarrhea; higher oral doses of loperamide (4 mg in the morning and 2 mg after each bowel movement) or diphenoxylate (5 mg tid or qid), deodorized opium tincture 0.5 to 0.75 mL (10 to 15 drops) q 4 to 6 h, or codeine 15 to 30 mg q 4 to 6 h may be required for more intense diarrhea. These antidiarrheal drugs must be used with extreme caution in more severe cases because they may precipitate toxic dilation.

In mild or moderate disease, treatment may sometimes be achieved with a hydrocortisone enema instead of with oral corticosteroid therapy. Mesalamine may also be given by enema and is beneficial in many cases of refractory proctosigmoiditis and left-sided colitis. More extensive mild or moderate disease as well as localized disease may respond to oral sulfasalazine. Once remission is achieved, long-term maintenance therapy with sulfasalazine 1 to 3 g/day is indicated to prevent relapse.

Moderately severe disease in ambulatory patients usually requires systemic corticosteroid therapy. Severe disease, manifested by >10 bloody bowel movements per day, tachycardia, high fever, or severe abdominal pain, requires hospitalization. If the patient had been receiving corticosteroid treatment ≧30 days at the time of admission, hydrocortisone 300 mg/day should be given by continuous IV drip. Oral prednisone 60 mg/day may be substituted after remission has been achieved with the 7- to 10-day course of parenteral treatment. A patient who remains well on the oral regimen for 3 to 4 days may leave the hospital, and the corticosteroid dosage may be gradually reduced at home under-close medical supervision.

Immunomodulatory drugs are acceptable for some patients with refractory or corticosteroid-dependent ulcerative colitis. Azathioprine and 6-mercaptopurine inhibit T-cell function, and a decline in the activity of both natural killer cells and cytotoxic T cells is correlated with a clinical response. Cyclosporine has a rapid onset and is primarily indicated for acute severe ulcerative colitis unresponsive to high-dose IV corticosteroids.

Pressure Sores (Skin Ulcers). Pressure sores (bedsores; decubitus ulcers; trophic ulcers) can result from ischemic necrosis and ulceration of tissues overlying a bony prominence that has been subjected to prolonged pressure. Pressure sores occur most often in patients with diminished or absent sensation or who are debilitated, emaciated, paralyzed, or long bedridden. Tissues over the sacrum, ischia, greater trochanters, external malleoli, and heels are especially susceptible; other sites may be involved depending on the patient's position. Pressure sores can also affect muscle and bone. The ulcer is analogous to an iceberg; it has a small visible surface with a more extensive unknown base.

Deep Venous Ulcers (Skin Ulcers). Deep venous ulcers are the most common type of ulcer affecting the lower extremities. The normal vein has valves that prevent the backflow of blood. When these valves become incompetent, the backflow of venous blood causes venous congestion leading to obstruction of the normal vascularity of the area and necrosis of the muscle depending on the compromised vascular supply. The typical venous ulcer appears near the medial malleolus, is in combination with an edematous and indurated lower extremity, is shallow, is not too painful and may present with a weeping discharge from the leg. Treatment of venous ulcers are directed at keeping the ulcer infection free, absorbing any excess discharge, maintaining a moist wound environment, supplying compression, promoting activity of the patient and the involved extremity and managing the patient's medical problems. Controlling the edema is a primary concern.

The complexity of the biological processes involved in the generation and repair of ulcers is not fully appreciated, but a central role for inflammation in both acute and chronic stages of ulcers is recognized. In both humans and animal models of disease, ulcers develop as a result of sequential interaction of epithelial cells and subepithelial tissues with pro-inflammatory cytokines and local factors. While epithelial degeneration alone qualifies as ucleration, the clinical terminology includes deeper penetration into the subepithelial environment (soft tissue, vasculature and muscle). (National Digestive Disorders Information Clearinghouse, at www.niddk.nih.gov) (Pathology, Rubin E and Fara J L, eds. JB Lippincott Co, 2^{nd} Edition, 1994). The inflammatory process initiates in subepithelial layers and proceeds toward the surface epithelium. Other types of wound first destroy the epithelium, and subsequently activate an inflammatory reaction that has the potential to destroy the subepithelial layers (e.g. as scald, chemical or electrical bums, incisions and punctures.

Dehydroepiandrosterone (DHEA; (3β)-3-hydroxyandrost-5-en-17-one) is a weak androgen that serves as the primary precursor in the biosynthesis of both androgens and estrogens (De Peretti et al., 1978). DHEA has been reported to play a mitigating role in obesity, diabetes, carcinogenesis, autoimmunity, neurological loss of memory (Schwartz et al., 1981; Yen et al., 1977; Coleman, 1982; Flood, 1988), and the negative effects of GCS on IL-2 production by murine T cells (Daynes et al., 1990). Araneo et al. (1993) has shown that the administration of DHEA to burned mice within one hour after injury resulted in the preservation of normal immunologic competence, including the normal capacity to produce T-cell-derived lymphokines, the generation of cellular immune responses and the ability to resist an induced infection. Eich et al. (1992a; 1992b) describes the use of DHEA to reduce the rate of platelet aggregation and the use of DHEA or DHEA sulfate (DHEAS) to reduce the production of thromboxane, respectively.

Nestler et al. (1990) shows that administration of DHEA was able in human patients to reduce body fat mass, increase muscle mass, lower LDL cholesterol levels without affecting HDL cholesterol levels, lower serum apolipoprotein B levels, and not affect tissue sensitivity to insulin. Kent (1982) reported DHEA to be a “miracle drug” which may prevent obesity, aging, diabetes mellitus and heart disease. DHEA was widely prescribed as a drug treatment for many years. However, the Food and Drug Administration recently restricted its use. DHEA is readily interconvertible with its sulfate ester DHEAS through the action of intracellular sulfatases and sulfotransferases.

Daynes et al. (1994) shows that administration of certain DHEA derivatives is useful for the reducing or preventing progressive tissue necrosis following scald bum. DHEA is useful in the prevention of bacterial translocation through intact intestinal membranes and adult respiratory distress syndrome. It has also been discovered that burn-injured mice that are treated within one hour after thermal injury with DHEA develop and resolve their burn wounds sooner and faster than untreated or sham treated thermally injured controls. Daynes et al. (1996) shows that the administration of DHEAS and other DHEA derivatives are also suitable for these uses. Araneo et al. (1998) shows that DHEA derivatives are useful for reducing or preventing pulmonary hypertension. Araneo (1999) also shows that DHEA and derivatives are useful for enhancing re-epithelialization or re-endoehtlialization; a process that restores damaged or lost outermost layers of the skin or mucous membranes, where the injury has a specific causal event, a situation unlike ulcers which have no specific causation. Finally, there has been a report (Andus et al., 2000) of the treatment of ulcerative colitis by administering DHEA in an amount which restores DHEAS blood levels to low normal levels.

Despite the myriad of biological activities reported for DHEA derivatives, DHEA derivatives have not been reported to have any affect on prevention and/or treatment of ulcers.

### SUMMARY OF THE INVENTION

The present invention is related to therapeutic uses of dehydroepiandrosterone (DHEA) congeners or metabolites thereof (also collectively referred to as DHEA therapeutic) as active agents in the prevention and/or treatment of ulcers. In a preferred embodiment, the active agent is DHEA. These uses include methods for treating and/or preventing the development of ulcers in skin and mucosal tissues. In accordance with the present invention, the administration of a therapeutic amount of a DHEA congener or a metabolite thereof to a patient in need thereof or a patient at risk for developing such ulcers accomplishes these methods.

In a first embodiment, the invention provides a method for reducing or preventing the severity and duration of ulcers in a subject at risk for ulceration, which comprises the administration of a DHEA congener or metabolite thereof as an active agent prior to appearance of ulcers. This is similar to treatment of patients to maintain remission of ulcers. The DHEA therapeutic is administered in a manner and dose that causes alleviation and/or prevention of ulcers and symptoms employing routine pharmnacologic practices. In a preferred embodiment, the active agent is DHEA.

In a second embodiment, the invention provides a method of treating an ulcer, which comprises administering a DHEA congener or metabolite thereof to a subject having an ulcer or ulcerative condition to promote rapid repair and regeneration of the tissue. The ulcer or condition can be any type of skin or mucosal ulcer and involve only the epithelium or can include its surrounding subepithelial tissue and the vascular supply. In a preferred embodiment, the active agent is DHEA.

In a third embodiment, the invention provides a method of preventing or reducing weight loss, cachexia, which comprises administering a DHEA congener or metabolite thereof to a subject in need thereof. In a preferred embodiment weight loss may be induced by factors associated with a primary therapy for the subject (e.g., radiation or chemotherapy for cancer). In a further preferred embodiment, weight loss may result from systemic inflammation caused by the ulcer. Weight loss may be mild or moderate, and/or of short or long duration. In a preferred embodiment, the active agent is DHEA.

### BRIEF DESCRIPTION OF THE FIGURES

FIG. 1 is an illustration of normal skin.

FIG. 2 is an illustration of ulceration of the skin.

FIG. 3 is an illustration of normal intestinal mucosa.

FIG. 4 is an illustration of ulcerated intestine.

FIGS. 5A-5B are graphic presentations showing the effects of DHEA on weight change following irradiation.

FIGS. 6A-6D are graphic presentations showing the effects of DHEA on the development of mucositis following irradiation.

FIG. 7 is a graphic presentation showing the effects of DHEA on weight loss in a colitis model.

FIG. 8 is a graphic presentation showing the effects of DHEA on the development of colon injury in a colitis model.

FIG. 9 is a graphic presentation showing the effects of DHEA on colon weight change in a colitis model.

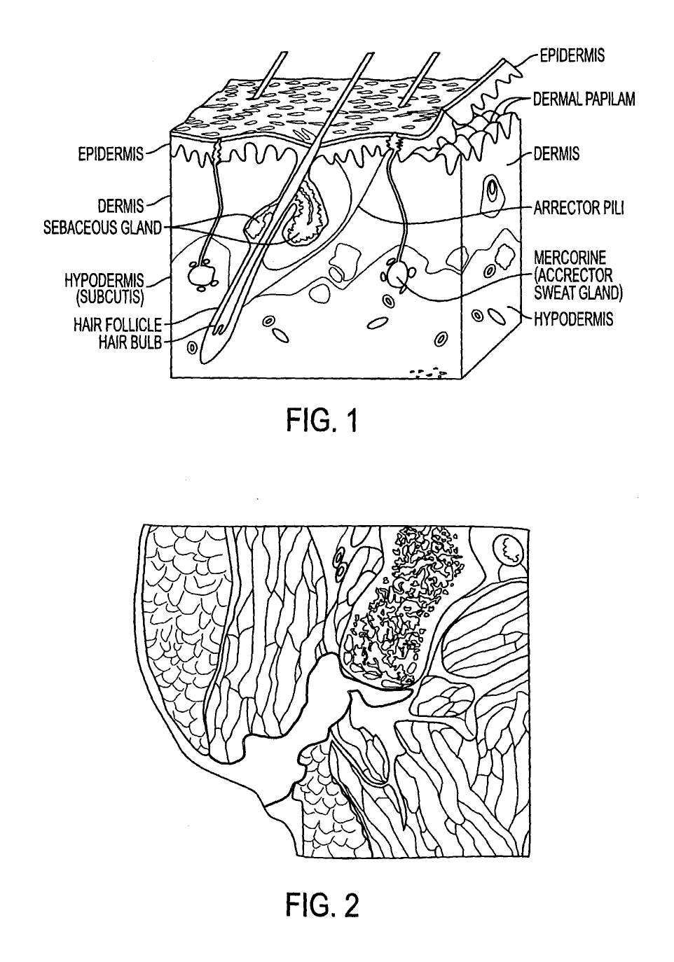

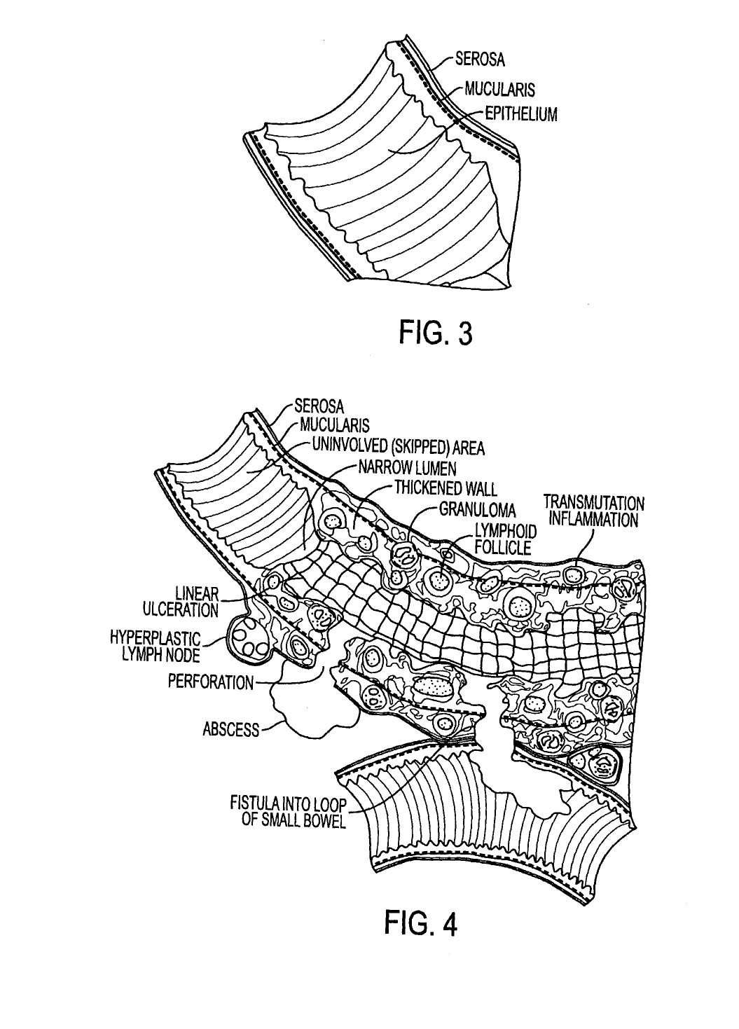

### DETAILED DESCRIPTION OF THE INVENTION

In one aspect of the present invention, a method is provided for preventing and/or treating ulcers. The ulcers may be ulcers in skin or mucosal tissues, such as described above. In accordance with this aspect of the invention, tissue shows reduced ulceration if a DHEA congener or metabolite thereof is administered in a therapeutic amount. A therapeutic amount is a dosage that achieves the therapeutic index in a subject in need or at risk.

Examples of a DHEA congener include, but are not limited to, compounds having the general formula I and their pharmaceutically acceptable salts:

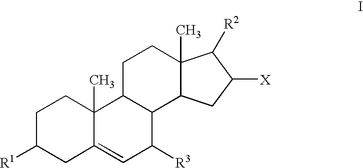

wherein

X is H or halogen;

R¹, R² and R³ are independently ═O, —OH, —SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R⁴ or —OPOR⁴R⁵;

R⁴ and R⁵ are independently —OH, pharmaceutically acceptable esters or pharmaceutically acceptable ethers.

Suitable metabolites of DHEA include, but are not limited to, dehydroepiandrosterone sulfate, 16α-hydroxydehydroepiandrosterone, 16α-hydroxyandrost-4-ene-3,17dione, androst-4-ene-3,17 dione, 7α-hydroxyandrostenedione, 7α-hydroxytestosterone.

Further examples of a DHEA congener, include but are not limited to, compounds having the general formulas II and III and their pharmaceutically acceptable salts:

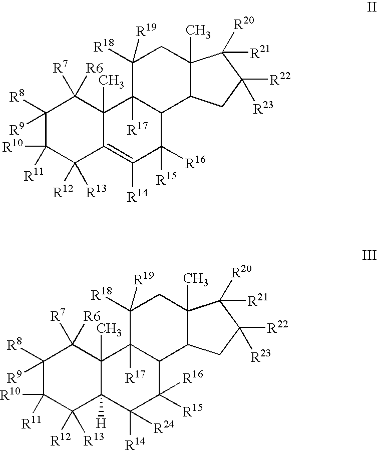

wherein

R⁶, R⁷, R⁸, R⁹, R¹¹, R¹², R¹³, R¹⁴, R¹⁵, R¹⁶, R¹⁷, R¹⁸, R¹⁹ and R²⁴ are independently H, —OH, halogen, C₁₋₁₀ alkyl or C₁₋₁₀ alkoxy;

R¹⁰ is H, —OH, halogen, C₁₋₁₀ alkyl, or C₁₋₁₀ alkoxy;

R²⁰ is (1) H, halogen, C₁₋₁₀ alkyl or C₁₋₁₀ alkoxy when R²¹ is —C(O)OR²⁵ or

(2) H, halogen, OH or C₁₋₁₀ alkyl when R²¹ is H, halogen, OH or C₁₋₁₀ alkyl or

(3) H, halogen, C₁₋₁₀ alkyl, C₁₋₁₀ alkenyl, C₁₋₁₀ alkynyl, formyl, C₁₋₁₀ alkanoyl or epoxy when R²¹ is OH; or

R²⁰ and R²¹ taken together are ═O;

R²² and R²³ are independently (1) H, —OH, halogen, C₁₋₁₀ alkyl or C₁₋₁₀ alkoxy when R²¹ is H, OH, halogen, C₁₋₁₀ alkyl or —C(O)OR²⁵ or (2) H, (C₁₋₁₀ alkyl)ₙamino, (C₁₋₁₀ alkyl)ₙamino-C₁₋₁₀ alkyl, C₁₋₁₀ alkoxy, hydroxy-C₁₋₁₀ alkyl, C₁₋₁₀ alkoxy-C₁₋₁₀ alkyl, (halogen)ₘ-C₁₋₁₀ alkyl, C₁₋₁₀ alkanoyl, formyl, C₁₋₁₀ carbalkoxy or C₁₋₁₀ alkanoyloxy when R²⁰ and R²¹ taken together are ═O; or

R²² and R²³ taken together are ═O or taken together with the carbon to which they are attached form a 3-6 member ring containing 0 or 1 oxygen atom; or

R²⁰ and R²² taken together with the carbons to which they are attached form an epoxide ring;

R²⁵ is H, (halogen)ₘ-C₁₋₁₀ alkyl or C₁₋₁₀ alkyl;

n is 0, 1 or 2;

m is 1, 2 or 3; and

physiologically acceptable salts thereof,

with the provisos that

(a) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ and R²¹ taken together are ═O; and

(b) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ is H and R²¹ is H, OH or halogen.

The compounds represented by the general formula I exist in many stereoisomers and the formula is intended to encompass the various stereoisomers. Examples of suitable DHEA congeners of Formula I include compounds in which:

(1) R² is ═O, R³ and X are each H and R¹ is ═O, —OH, pharmaceutically acceptable esters thereof, pharmaceutically acceptable ethers thereof or pharmaceutically acceptable salts;

(2) R² is ═O, R³ is H, X is halogen and R¹ is ═O, —OH, pharmaceutically acceptable esters thereof, pharmaceutically acceptable ethers thereof or pharmaceutically acceptable salts;

(3) R² is ═O, R³ and X are each H and R¹ is —SH, pharmaceutically acceptable thioesters thereof, pharmaceutically acceptable thioethers thereof or pharrnaceutically acceptable salts;

(4) R² is ═O, R³ is H, X is halogen and R¹ is —SH, pharmaceutically acceptable thioesters thereof, pharmaceutically acceptable thioethers thereof or pharmaceutically acceptable salts;

(5) R² is ═O, X is H and R¹ and R³ are independently ═O, —OH, pharmaceutically acceptable esters thereof, pharmaceutically acceptable ethers thereof or pharmaceutically acceptable salts;

(6) R² is ═O, X is halogen and R¹ and R³ are independently ═O, —OH, pharmaceutically acceptable esters thereof, pharmaceutically acceptable ethers thereof or pharmaceutically acceptable salts;

(7) R² is ═O, X is H and R¹ and R³ are independently —SH, pharmaceutically acceptable thioesters thereof, pharmaceutically acceptable thioethers thereof or pharmaceutically acceptable salts;

(8) R² is ═O, X is halogen and R¹ and R³ are independently —SH, pharmaceutically acceptable thioesters thereof, pharmaceutically acceptable thioethers thereof or pharmaceutically acceptable salts;

(9) R² is —OH, R³ and X are each H and R¹ is ═O, —OH, pharmaceutically acceptable esters thereof, pharmaceutically acceptable ethers thereof or pharmaceutically acceptable salts;

(10) R² is —OH, R³ is H, X is halogen and R¹ is ═O, —OH, pharmaceutically acceptable esters thereof, pharmaceutically acceptable ethers thereof or pharmaceutically acceptable salts;

(11) R² is —OH, R³ and X are each H and R¹ is —SH, pharmaceutically acceptable thioesters thereof, pharmaceutically acceptable thioethers thereof or pharmaceutically acceptable salts;

(12) R² is —OH, R³ is H, X is halogen and R¹ is —SH, pharmaceutically acceptable thioesters thereof, pharmaceutically acceptable thioethers thereof or pharmaceutically acceptable salts;

(13) R² is —OH, X is H and R¹ and R³ are independently ═O, —OH, pharmaceutically acceptable esters thereof, pharmaceutically acceptable ethers thereof or pharmaceutically acceptable salts;

(14) R² is —OH, X is halogen and R¹ and R³ are independently ═O, —OH, pharmaceutically acceptable esters thereof, pharmaceutically acceptable ethers thereof or pharmaceutically acceptable salts;

(15) R² is —OH, X is H and R¹ and R³ are independently —SH, pharmaceutically acceptable thioesters thereof, pharmaceutically acceptable thioethers thereof or pharmaceutically acceptable salts;

(16) R² is —OH, X is halogen and R¹ and R³ are independently —SH, pharmaceutically acceptable thioesters thereof, pharmaceutically acceptable thioethers thereof or pharmaceutically acceptable salts;

(17) R² is —SH, R³ and X are each H and R¹ is ═O, —OH, pharmaceutically acceptable esters thereof, pharmaceutically acceptable ethers thereof or pharmaceutically acceptable salts;

(18) R² is —SH, R³ is H, X is halogen and R¹ is ═O, —OH, pharmaceutically acceptable esters thereof, pharmaceutically acceptable ethers thereof or pharmaceutically acceptable salts;

(19) R² is —SH, R³ and X are each H and R¹ is —SH, pharmaceutically acceptable thioesters thereof, pharmaceutically acceptable thioethers thereof or pharrnaceutically acceptable salts;

(20) R² is —SH, R³ is H, X is halogen and R¹ is —SH, pharmaceutically acceptable thioesters thereof, pharmaceutically acceptable thioethers thereof or pharmaceutically acceptable salts;

(21) R² is —SH, X is H and R¹ and R³ are independently ═O, —OH, pharmaceutically acceptable esters thereof, pharmaceutically acceptable ethers thereof or pharmaceutically acceptable salts;

(22) R² is —SH, X is halogen and R¹ and R³ are independently ═O, —OH, pharmaceutically acceptable esters thereof, pharmaceutically acceptable ethers thereof or pharmaceutically acceptable salts;

(23) R² is —SH, X is H and R¹ and R³ are independently —SH, pharmaceutically acceptable thioesters thereof, pharmaceutically acceptable thioethers thereof or pharmaceutically acceptable salts;

(24) R² is —SH, X is halogen and R¹ and R³ are independently —SH, pharmaceutically acceptable thioesters thereof, pharmaceutically acceptable thioethers thereof or pharmaceutically acceptable salts;

(25) X is H and R¹ is ═O, —OH, pharmaceutically acceptable esters thereof pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;

(26) X is halogen and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;

(27) X is H, R¹ is ═O or —OH, and R² and R³ are independently ═O, —OH, pharmaceutically acceptable inorganic esters thereof or pharmaceutically acceptable salts, wherein at least one of R² and R³ is an inorganic ester;

(28) X is halogen R¹ is ═O or —OH, and R² and R³ are independently ═O, —OH, pharmaceutically acceptable inorganic esters thereof or pharmaceutically acceptable salts, wherein at least one of R² and R³ is an inorganic ester.

Pharmaceutically acceptable esters or thioesters include, but are not limited to, esters or thioesters of the formula —OOCR or —SOCR, wherein R is a pharmaceutically acceptable alkyl, alkenyl, aryl, alkylaryl, arylalkyl, sphingosine or substituted sphingotipid groups, such as propionate, enanthate, cypionate, succinate, decanoate and phenylpropionate esters.

Pharmaceutically acceptable ethers or thioethers include, but are not limited to, ethers or thioethers of the formula —OR or —SR, wherein R is as defined above or enol, or —OR is an unsubstituted or substituted spirooxirane or —SR is a spirothiane.

Suitable sugar residues include, but are not limited to monosaccharides, disaccharides and oligosaccharides, such as a glucuronate.

Pharmaceutically acceptable inorganic esters include, but are not limited to, inorganic esters of the formula —OSO₂R⁴ or —OPOR⁴R⁵, wherein R⁴ and R⁵ are independently —OH, pharmaceutically acceptable esters, pharmaceutically acceptable ethers or pharmaceutically acceptable salts.

Compounds of general formulas II and III are synthesized as described in U.S. Pat. Nos. 4,898,694; 5,001,119; 5,028,631; and 5,175,154, incorporated herein by reference. The compounds represented by the general formulas II and III exist in many stereoisomers and these formulas are intended to encompass the various stereoisomers. Examples of representative compounds, which fall within the scope of general formulas II and III, include the following:

5α-androstan-17-one;

16α-fluoro-5α-androstan-17-one;

3β-methyl-5α-androsten-17-one;

16β-fluoro-5α-androstan-17-one;

17β-bromo-5-androsten-16-one;

17β-fluoro-3β-methyl-5-androsten-16-one;

17α-fluoro-5α-androstan-16-one;

3β-hydroxy-5-androsten-17-one;

17α-methyl-5α-androstan-16-one;

16α-methyl-5-androsten-17-one;

3β,16α-dimethyl-5-androsten-17-one;

3β,17α-dimethyl-5-androsten-16-one;

16α-hydroxy-5-androsten-17-one;

16α-fluoro-16β-methyl-5-androsten-17-one;

16α-methyl-5α-androstan-17-one;

16-dimethylaminomethyl-5α-androstan-17-one;

16β-methoxy-5-androsten-17-one;

16α-fluoromethyl-5-androsten-17-one;

16-methylene-5-androsten-17-one;

16-cyclopropyl-5α-androstan-17-one;

16-cyclobutyl-5-androsten-17-one;

16-hydroxymethylene-5-androsten-17-one;

3α-bromo-16α-methoxy-5-androsten-17-one;

16-oxymethylene-5-androsten-17-one;

3β-methyl-16ξ-trifluoromethyl-5α-androstan-17-one;

16-carbomethoxy-5-androsten-17-one;

3β-methyl-16β-methoxy-5α-androstan-17-one;

3β-hydroxy-16α-dimethylamino-5-androsten-17-one;

17α-methyl-5-androsten-17β-ol;

17α-ethynyl-5α-androstan-17β-ol;

17β-formyl-5α-androstan-17β-ol;

20,21-epoxy-5α-pregnan-17α-ol;

3β-hydroxy-20,21-epoxy-5α-pregnan-17α-ol;

16α-fluoro-17α-ethenyl-5-androsten-17β-ol;

16α-hydroxy-5-androsten-17α-ol;

16α-methyl-5α-androstan-17α-ol;

16α-methyl-16β-fluoro-5α-androstan-17α-ol;

16α-methyl-16β-fluoro-3-hydroxy-5-androsten-17α-ol;

3β,16β-dimethyl-5-androsten-17β-ol;

3β,16,16-trimethyl-5-androsten-17β-ol;

3β,16,16-trimethyl-5-androsten-17-one;

3β-hydroxy-4α-methyl-5-androsten-17α-ol;

3β-hydroxy-4α-methyl-5-androsten-17-one;

3α-hydroxy-1α-methyl-5-androsten-17-one;

3α-ethoxy-5α-androstan-17β-ol;

5α-pregnan-20-one;

3β-methyl-5α-pregnan-20-one;

16α-methyl-5-pregnen-20-one;

16α-methyl-3β-hydroxy-5-pregnen-20-one;

17α-fluoro-5-pregnen-20-one;

21-fluoro-5α-pregnan-20-one;

17α-methyl-5-pregnen-20-one;

20-acetoxy-cis-17(20)-5α-pregnene;

3α-methyl-16,17-epoxy-5-pregnen-20-one.

The effectiveness of DHEA in reducing ulcers has been examined in animal systems that are widely used as models for human disease. In one aspect, for example, the animal system can comprise a rat or hamster model of ulceration. These models are highly reproducible and achieve ulceration with soft tissue involvement; e.g., oral mucositis as representative of oral pharyngeal ulcers and colitis as representative of gastrointestinal ulcerative diseases in humans. The administration of DHEA in these models demonstrates that DHEA can both treat and prevent ulceration. Thus, this model demonstrates the ability of DHEA congeners for the treatment and/or prevention of ulcerative conditions such as mucosal ulcers including oral, peptic, intestinal, colonic and rectal ulcers and skin ulcers including both surface and deep venous ulcers. The animal models used to describe the inventions herein are equally predictive of their effect in human ulcers.

Thus, the present invention provides methods for treating and/or preventing the development of ulcers in skin and mucosal tissues. In accordance with the present invention, these methods are accomplished by administering a therapeutic amount of a DHEA congener or a metabolite thereof to a patient in need thereof or a patient at risk for developing such ulcers.

In a first embodiment, the invention provides a method for reducing or preventing the severity and duration of ulcers in a subject at risk for ulceration, which comprises prophylactic administration of a DHEA congener or metabolite thereof as an active agent prior to appearance of ulcers. This is similar to treatment of patients to reduce or ameliorate ulcer symptoms. The DHEA therapeutic is administered in a manner and dose that causes alleviation and/or prevention of ulcers and symptoms employing routine pharmacologic practices. In a preferred embodiment, the active agent is DHEA.

In a second embodiment, the invention provides a method of treating an ulcer, which comprises therapeutic administration of a DHEA congener or metabolite thereof to a subject having an ulcer or ulcerative condition to promote induction of remission of ulcer symptoms. The ulcer or condition can be any type of skin or mucosal ulcer and involve only the epithelium or can include its surrounding subepithelial tissue and the vascular supply. In a preferred embodiment, the active agent is DHEA.

In a third embodiment, the invention provides a method of preventing or reducing weight loss, cachexia, which comprises administering a DHEA congener or metabolite thereof to a subject in need thereof. In a preferred embodiment weight loss may be induced by factors associated with a primary therapy for the subject (e.g., radiation or chemotherapy for cancer). In a further preferred embodiment, weight loss may result from systemic inflammation caused by the ulcer. Weight loss may be mild or moderate, and/or of short or long duration. In a preferred embodiment, the active agent is DHEA.

Pharmaceutical compositions containing a compound of the present invention as the active ingredient can be prepared according to conventional pharmaceutical compounding techniques (Remington's Pharmaceutical Sciences). Typically, a therapeutically effective amount of the active ingredient will be admixed with a pharmaceutically acceptable carrier. The carrier may take a wide variety of forms depending on the form of preparation desired for administration, e.g., oral, intravenous, intrathecal and epidural, transdermal, transbuccal, ocular, nasal, suppository. It is preferred to administer the active agent by oral administration for subacute and chronic treatment and/or prevention of ulcers in humans. The compositions may further contain antioxidizing agents, stabilizing agents, preservatives and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, solubilizing or suspending agents, and the like in the case of oral liquid preparations (such as, for example, suspensions, elixirs and solutions); or carriers such as starches, sugars, bicarbonates, diluents, granulating agents, lubricants, binders, disintegrating and dispersing agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698, incorporated herein by reference.

To achieve a therapeutic level in systemic circulation, the compound (i.e., active agent) is formulated using standard techniques to form a composition having a high bioavailability of the active agent in order to meet the desired therapeutic blood levels. The active agent, a complex of the active agent and a cyclodextrin or the active agent in a nanoparticle delivery system may be dissolved in a pharmaceutical carrier and administered as either a solution or a suspension. Cyclodextrins of all classes (alpha, beta and gamma) and their substituted or derivatized forms can be used, as well as mixtures thereof. It is preferred to use a complex of the active agent with a cyclodextrin or the active agent in a nanoparticle delivery system. It is especially preferred to use a complex of the active agent and a cyclodextrin, preferably 2-hydroxypropyl β-cyclodextrin, such as prepared in accordance with U.S. Pat. No. 4,727,064 and/or European Patent No. 0 149 197, each incorporated herein by reference. The use of the compound as part of a cyclodextrin complex or nanoparticle delivery system allows for the preparation of both parenteral and oral solutions and oral solid dosage forms with high concentration of active agent. Illustrative of suitable carriers for complexes of the active agent and the excipient are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they or their cyclodextrin complexes or nanoparticle delivery systems may also be dissolved in cerebrospinal fluid.

The active agent is preferably administered in a therapeutically effective amount. The actual amount administered, and the rate and time-course of administration, will depend on the nature and severity of the condition being treated. Thus, it may be desirable to administer the highly bioavailable complex of the active agent at several intervals during a 24-hour day to maintain blood levels at the therapeutic index. It would be preferable to administer the formulated active agent two to four times per day, and more preferably to administer it two to three times per day. Alternatively, one might use one of the oral controlled release methods to meter the drug available for absorption over a 24 hour period thereby necessitating only a single dose per day. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

The dose of the active agent is based on well known pharmaceutically acceptable principles to deliver an equivalent dose of the active agent or congener. In one embodiment the active agent is DHEA. For unprotected compounds, i.e., those which can be sulfated by human sulfotransferases or sulfatases, it is preferred to administer an excess dose to insure that sufficient active agent is administered, especially if sulfatases are not active at the site of administration. The dose of active agent will be administered between 1 and 4 times daily. In a preferred embodiment, the dose is administered twice daily and is DHEA.

As used herein, the terms “therapeutically effective amount” and “therapeutically effective dose” as applied to the active ingredient refers to the amount of the component in the composition or administered to the host that results in an increase in the therapeutic index of the host. The “therapeutic index” can be defined for purposes herein in terms of efficacy, i.e., extent of reduction or inhibition of ulcers. Suitable doses of the active ingredient can be determined using well-known methods, a variety of which are known and readily available in the pharmaceutical sciences, including, for example, measurement of markers associated with the disorder e.g., resistance to weight loss, decreased symptomatology, reduced pathology and remission of disease as examples. The dose of active agent delivered is selected to achieve DHEA or DHEA equivalent blood levels greater than normal endogenous DHEA blood levels. Normal endogenous blood levels of DHEA are less than 20 ng/ml. Accordingly, peak blood levels of DHEA or DHEA equivalent greater than 20 ng/ml are desired. Suitable doses are selected to achieve a peak blood level of DHEA or DHEA equivalent in the range from about 30 ng/ml to about 100 mg/ml, preferably in the range from about 50 ng/ml to about 10 mg/ml, more preferably in the range from about 100 ng/ml to about 1 mg/ml, even more preferably in the range from 100 ng/ml to about 100 μg/ml and most preferably from about 100 ng/ml to about 10 μg/ml.

As will be appreciated by those of skill in the art, the form of the pharmaceutical composition of the active agent and the mode of administration will determine the dose of the active agent to be delivered. An important factor in determining the proper dose to meet the desired peak blood levels is the bioavailability of the active agent in the pharmaceutical composition, i.e., the availability of the active agent for raising blood levels of DHEA or DHEA equivalent. The bioavailability is also dependent on the mode of administration. For example, the bioavailability of the active agent in a pharmaceutical composition delivered intravenously is greater than that for the same pharmaceutical composition delivered orally. Thus, a lower dose of a pharmaceutical composition containing the active agent can be administered intravenously than that which would be used orally. For oral administration of active agents with low water solubility, it is well recognized that co-formulation of the agent with a substance that accelerates dissolution or enhances solubility will increase the active agent's bioavailability. For example an active agent used to increase blood levels of DHEA or DHEA equivalent is over a thousand times more soluble in water if complexed with a cyclodextrin than without it. Similarly, the same active agent will dissolve in water much faster and have higher bioavailability if adsorbed to a high surface area particle with a large surface area than the uncomplexed agent. In view of these considerations, suitable blood levels of DHEA can be achieved, e.g., by the administration of 100 mg of a cyclodextrin-DHEA complex intravenously, or 600 mg of a cyclodextrin-DHEA complex orally or 500 mg of DHEA in a nanoparticle delivery system orally.

As will be appreciated by those of skill in the art, the preferred form of the pharmaceutical composition of the DHEA congener will depend on the intended mode of administration, which in turn will depend on the location and nature of the disorder to be treated. For example, delivery can be in the form of aqueous-based oral solutions, suspensions, emulsions, syrups, elixirs, gels, patches, lozenges, tablets, or capsules. Delivery to the gastrointestinal tract (e.g., for treatment of gastrointestinal mucositis, peptic ulcers and inflammatory bowel diseases, such as Crohn's disease, ulcerative colitis, indeterminate colitis, and infectious colitis) can be in the form of oral solutions, gels, suspensions, tablets, capsules, and the like.

Liquid carriers are used in preparing solutions, suspensions, emulsions, syrups, elixirs and pressurized compositions. The active ingredient can be dissolved or suspended in a pharmaceutically acceptable liquid carrier such as water, cyclodextrins, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral administration include water (partially containing solubilizers as above), alcohols (including monohydric alcohols and polyhydric alcohols, e.g., glycols) and their derivatives, oils (e.g., peanut oil, sesame oil, olive oil, and coconut oil), and combinations of the above. Compositions comprising such carriers and adjuvants may be formulated using well-known conventional materials and methods. Such materials and methods are described, for example, in Remington's Pharmaceutical Sciences.

A solid carrier can include one or more substances which may also act as flavoring agents, lubricants, solubilizers, suspending agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. The carrier and drug may form a single composite with drug adsorbed to its surface that effectively enhances the rate of dissolution in the gastrointestinal tract. The powders and tablet may contain up to 99% of the active ingredient, and may be formulated for immediate and/or sustained release of the active ingredient. Oral tablets may be prepared using a variety of well-known methods and in a variety of conventional forms.

In tablets, the active ingredient is mixed with a carrier having the necessary compression properties in suitable proportions and compacted in the shape and size desired. Exemplary forms include dry powder compaction tablets, micro-particulate systems (e.g., wherein the active ingredient is spray-dried onto a scaffold particle), and hard or soft-gel capsules. The tablets may be optionally covered with an enteric coating, which remains intact in the stomach, but will dissolve and release the contents of the tablet once it reaches the small intestine. Alternatively, the tablets may be formulated to enhance gastric uptake to avoid first pass effect in the liver following intestinal absorption. A number of commercially available coatings may be used depending on the target.

The composition may comprise one or more sustained or controlled release excipients such that a slow or sustained, preferably constant, release of the active ingredient is achieved. A wide variety of suitable excipients are known in the art and are not a part of this invention. Such sustained/controlled release excipients and systems are described, for example, in U.S. Pat. No. 5,612,053 (Baichwal et al.), U.S. Pat. No. 5,554,387 (Baichwal), U.S. Pat. No. 5,512,297 (Baichwal), U.S. Pat. No. 5,478,574 (Baichwal et al.), and U.S. Pat. No. 5,472,711 (Baichwal et al.), each of which is incorporated by reference herein. If desired, the pharmaceutical composition can be formulated to provide a pulse dose of the active ingredient. A variety of pulse-dose systems, which provide low or high-pulsed doses, are known in the art. In another embodiment of the invention, the pharmaceutical composition is formulated to provide direct and/or targeted delivery of the active ingredient to a specific anatomic site or sites within the gastrointestinal tract; e.g., the duodenum, jejunum, ileum, cecum and/or colon. Methods for providing targeted delivery of macromolecules, including proteins and polypeptides, to specific tissues or organs within a mammalian host are well known in the art.

### EXAMPLES

The present invention is described by reference to the following Examples, which are offered by way of illustration and are not intended to limit the invention in any manner. Standard techniques well known in the art or the techniques specifically described below were utilized.

### Example 1

### Effect of DHEA in Acute Radiation Model of Oral Mucositis

The acute radiation model in hamsters (see, e.g., Sonis et al., 2000) has proven to be an accurate, efficient and cost-effective technique to provide a preliminary evaluation of anti-mucositis compounds. The course of mucositis in this model is well defined and results in peak ulceration scores approximately 14-16 days following radiation. The acute model has been used to study specific mechanistic elements in the pathogenesis of mucositis. Molecules that show efficacy in the acute radiation model may be further evaluated in the more complex models of fractionated radiation, chemotherapy, or concomitant therapy. In this example, an acute radiation dose of 40 Gy on day 0 was administered and clinically significant mucositis was observed around day 14.

Animals. Male Golden Syrian hamsters were randomly and prospectively divided into six (6) treatment groups prior to irradiation as follows:

Group 1 Saline control, oral, bid; day −1 to day 27.

Group 2 Vehicle control, oral, bid; day −1 to day 27.

Group 3 25 mg/kg/day DHEA, ip; day −1 to day 27.

Group 4 50 mg/kg/day DHEA, ip; day −1 to day 27.

Group 5 250 mg/kg/day DHEA, po; day −1 to day 27.

Group 6 500 mg/kg/day DHEA, po; day −1 to day 27.

Hamsters were given an acute radiation dose directed to their oral mucosa. Test materials were applied by injection (once per day) or by oral dosing (either once or twice per day. Oral administrations were done using feeding needles. Dosing began the day before radiation (day −1) and continued until day 27. The test material utilized was an Inflabloc® pharmaceutical preparation, which, for the present example, comprised a complex of DHEA and cyclodextrin.

Starting on Day 6 and continuing every second day thereafter (days 8, 10, 12, 14, 16, 18, 20, 22, 24, 26, & 28), each animal was evaluated for mucositis scoring.

Mucositis Scoring. Parameters measured were the mucositis score, weight change and survival. For the evaluation of mucositis, the animals were anesthetized with inhalation anesthetics, and the left pouch everted. Mucositis was scored visually by comparison to a validated photographic scale, ranging from 0 for normal, to 5 for severe ulceration (clinical scoring).

A score of 1-2 is considered to represent a mild stage of the disease, whereas a score of 3-5 is considered to indicate moderate to severe mucositis. Following visual scoring, a photograph was taken of each animal's mucosa using a standardized technique. At the conclusion of the experiment, all film was developed and the photographs randomly numbered. At least two independent trained observers graded the photographs in blinded fashion using the above-described scale (blinded scoring).

The effect on mucositis of each drug treatment compared to placebo was assessed according to these parameters: the difference in the number of days hamsters in each group have severe (score 3) mucositis and the rank sum differences in daily mucositis scores. On each evaluation day, the number of animals with a blinded mucositis score of 3 in each drug treatment group, was compared to the control group. Differences were analyzed on a cumulative basis. For each evaluation day the scores of the control group were compared to those of the placebo group and vehicle group.

Mucositis Induction. Mucositis was induced using a standardized acute radiation protocol using a single dose of radiation (40 Gy/dose) on Day 0.

### RESULTS

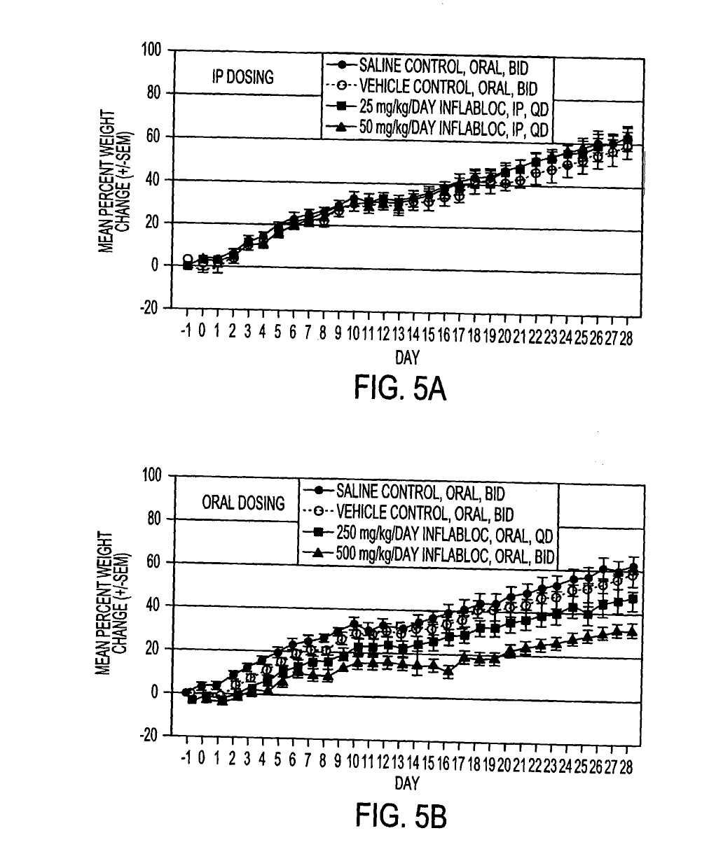

Weight Loss (FIG. 5). Groups treated by IP injection at 25 mg/kg/day or 50 mg/kg/day both showed weight gain patterns that closely corresponded to those observed in the control group. Groups treated 250 mg/kg/day or 500 mg/kg/day, p.o., both showed no pattern of weight loss.

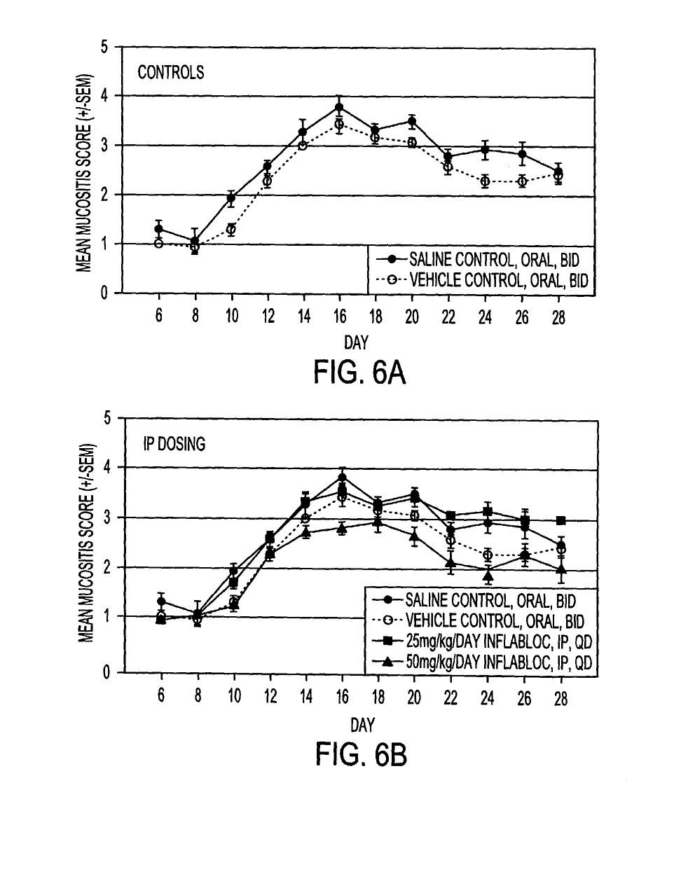

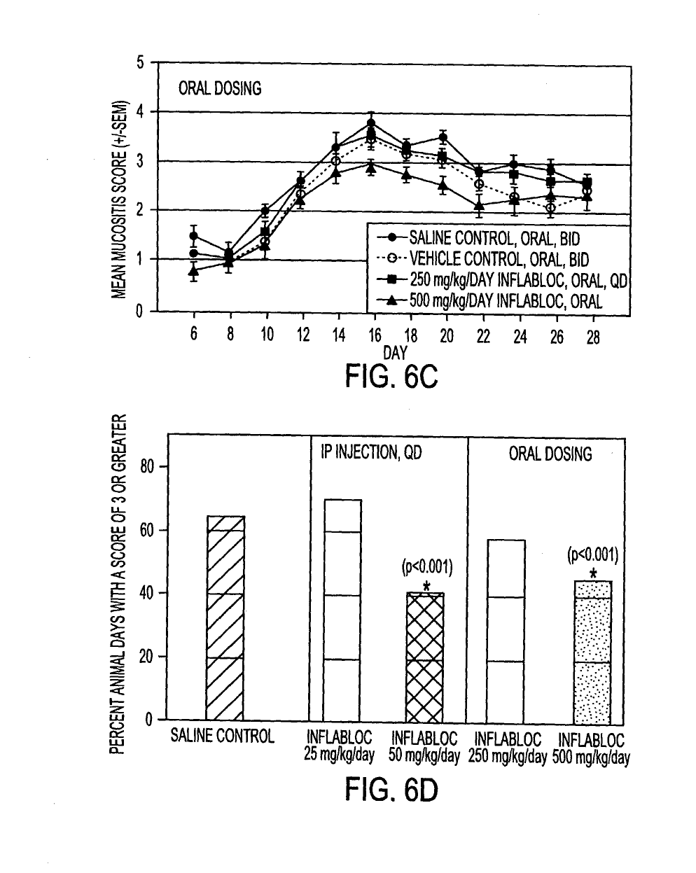

Mucositis (FIG. 6). Mean daily mucositis scores for each group are shown in FIGS. 6A-6D. The pattern of mucositis scores for the saline control animals is typical of the acute radiation model. Treatment with 25 mg/kg, i.p.,resulted in a pattern of mucositis severity that appeared similar to that observed in the saline control group while the scores for the group receiving 50 mg/kg DHEA were significantly lower than those in the saline control group for the entire study. Groups treated with 250 mg/kg/day DHEA resulted in a pattern of mucositis severity that appeared similar to that observed in the saline control group. The daily mucositis scores for the oral treatment group receiving 500 mg/kg/day DHEA were significantly lower than those in the saline control group.

### Example 2

### Effects of DHEA in TNBS-induced Model of Colitis

In this Example, the effects of DHEA were evaluated in the TNBS model (see, e.g., Boughton-Smith et al., 1988; Morris et al., 1989; Reuter et al., 1996) at a defined oral dose administered twice a day by gavage, using a low intracolonic concentration of TNBS (10 mg). This challenge dose of TNBS is known to generate reproducible yet not unduly severe mucosal injury in the colon, as determined 3 days after instillation. In this current investigation, colonic macroscopic injury has been assessed. In addition to DHEA, the effect of a single daily low oral dose of dexamethasone, which has a broad profile of anti-inflammatory actions, was also evaluated in this model.

Male Wistar rats (220-300 g) were randomised into groups before commencement of the study. On day 0, the rats were transiently anaesthetised with ether and TNBS (10 mg in 0.25 ml of 50% ethanol) was instilled into the colon using a soft plastic catheter inserted in the rat rectum. The rats were allowed to recover with free access to food and drinking water. At the end of the Experiment, 72 h after TNBS administration (i.e. on the morning of day 3), the distal colon was dissected, photographed and stored appropriately for subsequent analyses. Macroscopic scoring of distal 8 cm of colon was measured as the primary efficacy parameter. In addition, the weight of the colonic segment was assessed as an indirect and non-specific marker of oedema, and the body weight of the animals was determined each day of the study.

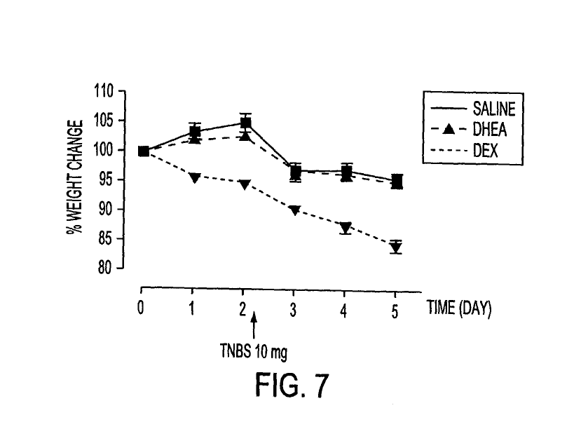

The groups (n=8-12) for study with TNBS challenge were: (a) saline control administered in 0.5 ml p.o. per rat, b.i.d; (b) DHEA (50 mg per rat, b.i.d); (c) dexamethasone (0.3 mg/kg, oral, once daily); (d) control non-challenged with TNBS and non-drug-treated (received only salineb.i.d.). The DHEA was administered orally, twice daily for 48 h before TNBS administration, on Day 0 with TNBS administration and on Day 1 and 2. The DHEA was administered as a solid bicarbonate composite providing high bioavailability. Tissues were removed 72 h after TNBS administration (Day 3). The effects on weight gain in the individual rats examined with this model are shown in FIG. 7. Saline treated animals on the final day were 95.6+/−1.7% of mean starting body weight. Similarly, DHEA-treated animals were 95.4+/−1.1% of their mean starting body weight. Weight loss in the Dexamethasone treatment group was significant at 84.5+/−1.3% of starting weight.

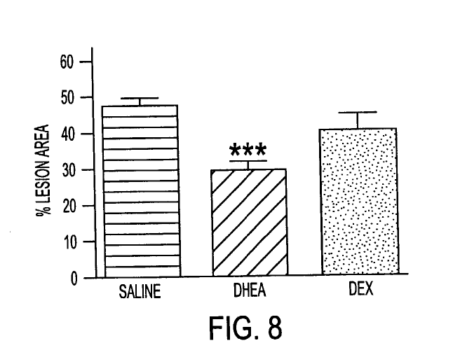

Macroscopic Colonic Injury. The area of colonic injury, determined 72 h after challenge in the control group of rats involved 48±2% (n=9) of the total colonic area of the segment studied, determined by computerized animetric measurement. There was no detectable macroscopic injury in the colons from non-challenged group of rats. Injury in DHEA treated animals involved 31+/−3% of the colon, a 40% reduction in the area of colonic injury (FIG. 8). This reduction in TNBS-induced colonic damage was statistically significant (P<0.001). In the dexamethasone-treated group (0.3 mg/kg once a day by the oral route), there was a small apparent reduction in macroscopic injury following TNBS challenge, with the damage involving 41±5% (n=8) of the mucosal area, but the extent of damage was not significantly different from the TNBS control group.

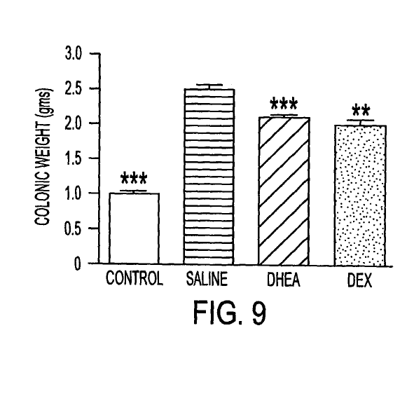

Colon Weight. As an indirect index of inflammatory oedema in the colonic tissue, the weight of the colonic segments was determined at the end of the study. As shown in FIG. 9, the colonic weight in the groups receiving saline and challenged with TNBS was significantly higher than that of non-challenged colon for a comparable tissue section. However, treatment with DHEA significantly (P<0.001) reduced the colonic weight of the standard segment and there was also a significant (P<0.01) reduction in colon weight in the dexamethasone group.

Oral administration of the DHEA preparation caused a significant fall in the extent of macroscopically assessed colonic damage, reducing the area of damage by some 40%.

The current findings thus demonstrate that the use of DHEA or its derivatives offer a novel approach to the therapy of the inflammatory bowel diseases, ulcerative colitis, Crohn's disease and other ulcerative conditions.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Andus, T. et al. (2000). Gastroenterology 118: A116.

Aranco, B. A. et al. (1993). Arch. Surg. 128:318-325.

Araneo, B. A. et al. (1998) U.S. Pat. No: 5,753,640.

Araneo, B. A. (1999). U.S. Pat. No. 5,929,060.

Boughton-Smith, N. K. et al. (1988). Br. J. Pharmacol. 94:65-72.

Coleman, D. C. (1982). Diabetes 31:830.

Daynes, R. A. et al. (1990). Eur. J. Immunol. 19:2319.

Daynes, R. A et al. (1994). WO 94/20111.

Daynes R. A. et al. (1996). U.S. Pat. No. 5,587,369.

De Peretti, E. et al. (1978). J. Clin. Endocrinol. Metab. 47:572.

Eich, D. M. et al. (1992a). U.S. Pat. No. 5,110,810.

Eich, D. M. et al. (1992b). U.S. Pat. No. 5,162,198.

Flood, J. F. (1988). Brain Res. 447:269.

Kent, S. (1982). Geriatrics 37:157-159.

Morris, G. P. et al. (1989). Gastroenterology 96:795-803.

Nestler, J. E. et al. (1990). U.S. Pat. No. 4,920,115.

Remington's Pharmaceutical Sciences, 18th Ed. (eds., A. F. Gennaro et al.), Mack Publishing Co., Easton, Pa. (1990).

Reuter, B. K. et al. (1996). J. Clin. Invest. 98:2076-2085.

Schwartz, A. G. et al. (1981). Nutr. Cancer 3:46.

Sonis, S. T. et al. (2000). Oral. Oncol. 96:373-381.

Yen, T. T. et al. (1977). Lipids 12:409.

European Patent No. 0 149 197.

PCT Published Application No. WO 96/11698.

U.S. Pat. No. 4,727,064.

U.S. Pat. No. 5,472,711.

U.S. Pat. No. 5,478,574.

U.S. Pat. No. 5,512,297.

U.S. Pat. No. 5,554,387.

U.S. Pat. No. 5,612,053.

What is claimed is:

1. A method for treating or preventing an ulcer which comprises administering to a subject at risk thereof or in need thereof an amount of an active agent selected from the group consisting of a dehydroepiandrosterone (DHEA) congener of Formula I, Formula II or Formula III, a metabolite of DHEA and pharmaceutically acceptable salts thereof effective to achieve a blood level of DHEA or DHEA equivalent greater than a normal endogenous blood level 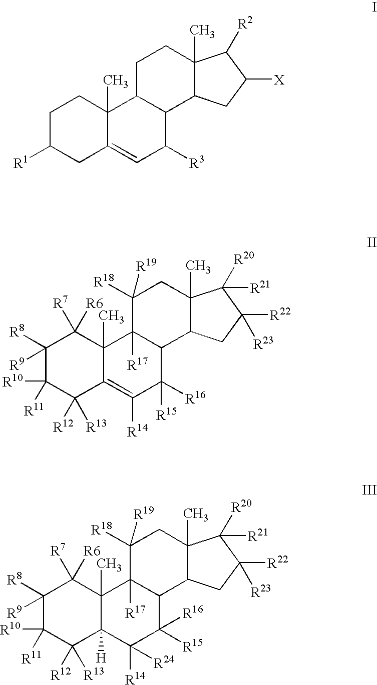
wherein
X is H or halogen;
R¹, R² and R³ are independently ═O, —OH, —SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R⁴ or —OPOR⁴R⁵;
R⁴ and R⁵ are independently —OH, pharmaceutically acceptable esters or pharmaceutically acceptable ethers;
R⁶, R⁷, R⁸, R⁹, R¹¹, R¹², R¹³, R¹⁴, R¹⁵, R¹⁶, R¹⁷, R¹⁸, R¹⁹ and R²⁴ are independently H, —OH, halogen, C₁₋₁₀ alkyl or C₁₋₁₀ alkoxy;
R¹⁰ is H, —OH, halogen, C₁₋₁₀ alkyl, or C₁₋₁₀ alkoxy;
R²⁰ is (1) H, halogen, C₁₋₁₀ alkyl or C₁₋₁₀ alkoxy when R²¹ is —C(O)OR²⁵ or
(2) H, halogen, OH or C₁₋₁₀ alkyl when R²¹ is H, halogen, OH or C₁₋₁₀ alkyl or
(3) H, halogen, C₁₋₁₀ alkyl, C₁₋₁₀ alkenyl, C₁₋₁₀ alkynyl, formyl, C₁₋₁₀ alkanoyl or epoxy when R²¹ is OH; or
R²⁰ and R²¹ taken together are ═O;
R²² and R²³ are independently (1) H, —OH, halogen, C₁₋₁₀ alkyl or C₁₋₁₀ alkoxy when R²¹ is H, OH, halogen, C₁₋₁₀ alkyl or —C(O)OR²⁵ or
(2) H, (C₁₋₁₀ alkyl)ₙamino, (C₁₋₁₀ alkyl)ₙamino-C₁₋₁₀ alkyl, C₁₋₁₀ alkoxy, hydroxy-C₁₋₁₀ alkyl; C₁₋₁₀ alkoxy-C₁₋₁₀ alkyl, (halogen)ₘ-C₁₋₁₀ alkyl, C₁₋₁₀ alkanoyl, formyl, C₁₋₁₀ arbalkoxy or C₁₋₁₀ alkanoyloxy when R²⁰ and R²¹ taken together are ═O; or
R²² and R²³ taken together are ═O or taken together with the carbon to which they are attached form a 3-6 member ring containing 0 or 1 oxygen atom; or
R²⁰ and R²² taken together with the carbons to which they are attached form an epoxide ring;
R²⁵ is H, (halogen)ₘ-C₁₋₁₀ alkyl or C₁₋₁₀ alkyl;
n is 0, 1 or 2; and
m is 1, 2 or 3;
with the provisos that (a) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH, or C₁₋₁₀ alkoxy and R²³ is H or halogen and R²⁰ and R²¹ taken together are ═O; and
(b) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ is H and R²¹ is H, OH or halogen;
whereby the ulcer is treated or prevented.

2. The method of claim 1, wherein the DHEA congener is a compound of Formula I.

3. The method of claim 1, wherein the DHEA metabolite is selected from the group consisting of dehydroepiandrosterone sulfate, 16α-hydroxydehydroepiandrosterone, 16α-hydroxyandrost-4-ene-3,17-dione, androst-4-ene-3,17-dione, 7α-hydroxyandrostene-dione and 7α-hydroxytestosterone.

4. The method of claim 1, wherein the DHEA congener is a compound of Formula II.

5. The method of claim 1, wherein the DHEA congener is a compound of Formula III.

6. The method of claim 1 wherein the active agent is administered in a pharmaceutical composition in the form of an oral solution, gel, suspension, pill, tablet, lozenge or capsule.

7. The method of claim 6 wherein the composition additionally contains single or multiple immediate release carrier compounds or delivery system for faster or more effective release of the active agent in the patient.

8. The method of claim 6 wherein the composition additionally contains a sustained release carrier for sustained release of the active agent in the gastrointestinal tract of the patient.

9. The method of claim 1, wherein the DHEA congener is DHEA.

10. The method of claim 1 wherein the active agent is administered orally.

11. The method of claim 10 wherein the DHEA congener is DHEA.

12. The method of claim 1 wherein the active agent is administered as a solid.

13. The method of claim 12 wherein the DHEA congener is DHEA.

14. The method of claim 1 wherein the active agent is administered as a liquid or a suspension.

15. The method of claim 14 wherein the DHEA congener is DHEA.

16. The method of claim 15 wherein the DHEA is administered as a formulation which comprises DHEA and a cyclodextrin selected from the group consisting of a cyclodextrin a substituted cyclodextrin, a derivatized cyclodextrin and a mixture thereof.

17. The method of claim 1 wherein the active agent is administered to the patient multiple times a day.

18. The method of claim 17 wherein the DHEA congener is DHEA.

19. The method of claim 1 wherein the ulcer causes a disease or disorder selected from the group consisting of mucositis, stomatitis, peptic and gastric ulcers, inflammatory bowel disease and skin or deep venous ulcers.

20. The method of claim 19 wherein the disease is inflammatory bowel disease.

21. The method of claim 20 wherein the inflammatory bowel disease is Crohn's disease.

22. The method of claim 20 wherein the inflammatory bowel disease is ulcerative colitis.

23. The method of claim 20 wherein the inflammatory bowel disease is indeterminate colitis.

24. The method of claim 20 wherein the inflammatory bowel disease is infectious colitis.

25. The method of claim 19 wherein the disease or disorder is mucositis.

26. The method of claim 19 wherein the disease or disorder is oral mucositis.

27. The method of claim 1, wherein the blood level is from about 30 ng/ml to about 100 mg/ml.

28. The method of claim 1, wherein the blood level is from about 50 ng/ml to about 10 mg/ml.

29. The method of claim 1, wherein the blood level is from about 100 ng/ml to about 1 mg/ml.

30. The method of claim 1, wherein the blood level is from about 100 ng/ml to about 100 μg/ml.

31. The method of claim 1, wherein the blood level is from about 100 ng/ml to about 10 μg/ml.
